(19) United States
(12) Patent Application Publication
(10) Pub. No.: US 2008/0039865 A1
(43) Pub. Date: Feb. 14, 2008

(54) Maasal cervical dilator

(76) Inventors: Maasal Shaher, Forest Park, IL (US); Ahmad Shaher, Forest Park, IL (US)

Correspondence Address: LADAS & PARRY LLP, 224 SOUTH MICHIGAN AVENUE, SUITE 1600, CHICAGO, IL, 60604, US

(21) Appl. No.: 11/655,529

(22) Filed: Jan. 19, 2007

Related U.S. Application Data
(60) Provisional application No. 60/836,337, filed on Aug. 8, 2006.

(51) Int. Cl. A61B 17/42; A61M 29/00

(52) U.S. Cl. 606/119; 606/191

(57) ABSTRACT

Opposing, contoured panels are controllably opened by either a translational or rotational movement of a driving control rod to controllably open or dilate a cervix. An insertion depth limiter, prevents over-insertion of the panels into the uterus thereby preventing accidental perforation of the uterine wall. The device can be straight, curved or articulated to accommodate anatomical differences.

### RELATED APPLICATIONS

This application claims the filing benefit of, and incorporates by reference, U.S. provisional patent application for “Maasal Dilator,” which was filed Aug. 8, 2006, and which is identified by patent application Ser. No. 60/836,337.

### BACKGROUND OF THE INVENTION

Many gynecological procedures require the cervix to be dilated or opened. In the prior art, cervical dilation is accomplished by inserting progressively larger-diameter probes, one-by-one into the cervix. A very small is inserted first, which causes the cervix to open by a correspondingly small amount. As is known, the cervix opens gradually.

After a very small probe is inserted, a slightly larger probe is inserted to cause the cervix to dilate further. One-by-one, progressively larger probes are inserted, with each probe causing the cervix to dilate further. By inserting increasingly larger diameter probes, the cervix is eventually dilated to the diameter required to access the interior of the uterus.

A problem with the prior art methods and prior art cervical dilation devices is the risk of perforating the uterine wall. Prior art cervical dilation probes can easily be inserted too far into the uterus, so far in fact that they can perforate the uterus. Moreover, cervical dilation using prior several-different diameter art probes, one-by-one is time-consuming. Cervical dilation using prior art probes can easily require more than twenty minutes to perform, prolonging the time required to perform an intra-uterine procedure.

A cervical dilator that reduces or eliminates the uterine wall perforation risk would be an improvement over the prior art. Moreover, a cervical dilator that can controllably open a cervix, i.e., cause it dilate, in less time than what is required using prior art methods would be an improvement. A cervical dilator that is able to open the cervix to a known diameter and which is also expandable while inside the cervix, would also be an improvement over the prior art.

### SUMMARY OF THE INVENTION

There is provided a cervical dilator referred to herein as a maasal cervical dilator, comprised of two or more elongated and contoured panels that form an expandable malecot that can be inserted into a cervix only a limited distance so as to prevent uterus wall perforation. Malecot insertion depth into the cervix 101 is limited by annular shoulder of a cylindrical sleeve that the expandable malecot extends past. The cylinder and its larger-than-the-cervix' inside diameter therefore acts a malecot insertion depth limiter.

In a fully retracted or collapsed state, the expandable malecot has an outside diameter that allows its insertion into the cervix. It its fully-expanded or opened state, the expandable malecot has an outside diameter significantly greater than the closed state of the cervix. The outside diameter of the shoulder is greater than the cervical diameter after dilation, such that the malecot cannot be inserted into the uterus.

The contoured panels that form the malecot are coupled to an elongated central control rod, which runs through the length of the panels and the insertion depth limiter. The control rod acts as part of a transmission mechanism, which converts linear or rotational movement of the control rod (or rotation of a thumb nut threaded onto the rod) into radial displacement of the contoured panels. In a preferred embodiment, the contoured panels are attached to the control rod by swing arms that are pivotally attached to both the control rod and the panels such that linear displacement of the control rod along its axis, causes the swing arm to fold outwardly, which in turn causes the panels to expand radially away from the control rod.

The distal end of the expandable malecot is rounded or blunted and smooth to facilitate cervix insertion and to avoid trauma to the cervix. In one embodiment, swing arms that enable expansion of the malecot panel can themselves be rounded to provide the rounded distal end.

### BRIEF DESCRIPTION OF THE DRAWINGS

FIG. 1 is a perspective view of an embodiment of cervical dilator with the malecot panels in their fully retracted position;

FIG. 2 is a perspective view of the cervical dilator of FIG. 1, but with the malecot panels in an extended position;

FIG. 2A is a schematic depiction of the swing arm and control geometry that effectuates radial displacement of the contoured panels by the linear displacement of the control rod;

FIG. 3 is a cross-sectional depiction of the cervix dilator in use;

FIG. 4. shows a cross-section of the malecot along sections lines 4-4 in FIG. 1, with the panels in their fully retracted position;

FIG. 5 shows a cross-section of the malecot along sections lines 4-4 in FIG. 1, with the panels in an extended position;

FIG. 6 is a length-wise cross-section of a cervix dilator, showing additional details of the transmission mechanisms

FIG. 7 shows a cervix dilator an alternate embodiment of a transmission mechanism, one that converts rotational movement into radial displacement;

FIGS. 8 and 9 show another embodiment of a cervix dilator wherein one panel is fixed and the second panel translates radially by movement of the control rod; and

FIGS. 10A, 10B, 11A, 11B and 12w depict alternate embodiments that use wavy or boustrophedonic surfaces to controllably displace panels away from each other.

### DETAILED DESCRIPTION

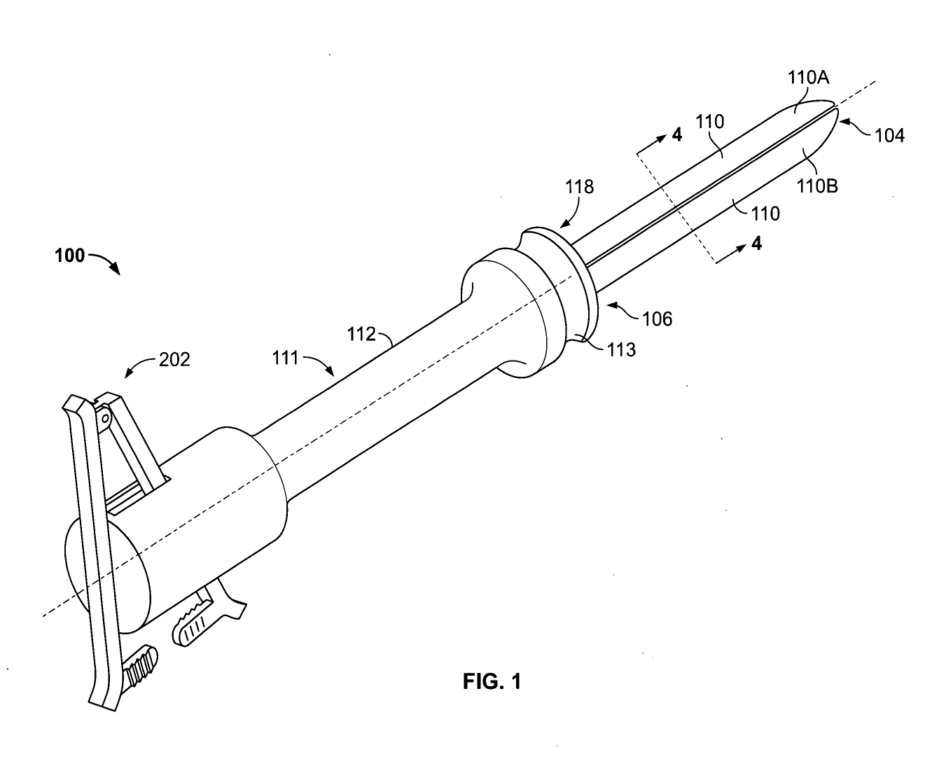

FIG. 1 shows a perspective view of a Maasal cervical dilator 100 according to one embodiment of the invention claimed herein. The dilator 100 is comprised of a thin and elongated dilating element embodied as dilating malecot 110 and an insertion depth limiter 112, which has an intermediate portion 111 that is narrowed to provide a grip for a user. The malecot 110 is in turn comprised of first and second elongated and contoured deflection panels 110A and 110B, which can be expanded away from each other as described below.

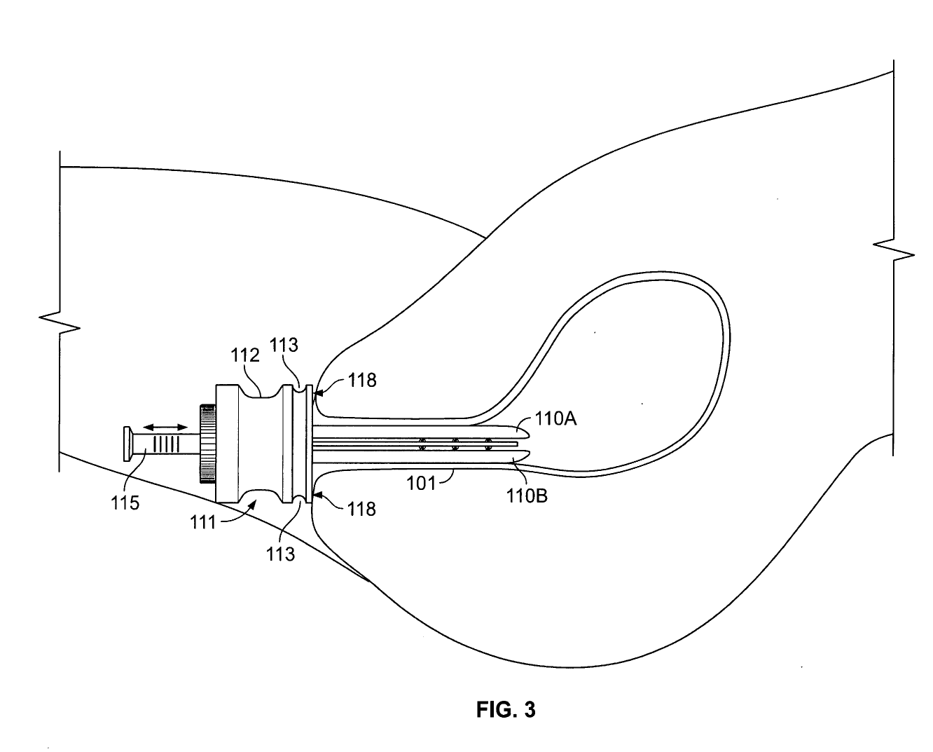

The malecot 110 has a rounded or tapered distal end 103, shaped as such to facilitate insertion of the malecot 110 and to avoid trauma to the cervix and cervical of the uterus as shown in FIG. 3. The rounded distal end 104 of the malecot is provided by rounding the distal end 104 of each of the contoured deflection panels 110A and 110B. In an alternate embodiment, a tapered and smoothened end is provided by modifying the distal set of the swing arms, 122, to form a tapered and smooth advancing end and articulate with the expanding malecots, 110.

The distal end 104 of the malecot 110 is separated from the proximal end 106 by a predetermined distance or length along a longitudinal axis 108 (not shown in FIG. 1) that runs through the center of the dilator 100. The distance between the proximal end 106 in the distal end 104 corresponds to the length of the malecot 110 that will extend into the cervical canal. The malecot's length is therefore preferably equal to or slightly less than the length of the cervical canal so that the entire length of the canal can be dilated, although malecots longer than and shorter than the cervix canal's length are also contemplated by the invention disclosed and claimed hereinafter. The longitudinal axis 108 along which the malecot's length is measured, can be considered to be a geometric center line that runs through the geometric center of the dilating element 102 as well as the insertion depth limiter 112, which is described below. In alternate embodiments, however, the longitudinal axis 108 can be off-center from the geometric center line.

The insertion depth limiter 112 acts as a handle for a user but it also importantly prevents the malecot 110 from being inserted too deeply. The narrowed portion 111 provides structure that improves the user's grip on the device. A circumferential groove 113 around the exterior surface of the insertion depth limiter 112 near the proximal end 106 of the malecot 110 is sized, shaped and arranged to accept the rim of the mouth of a prophylactic cover, not shown in the drawings.

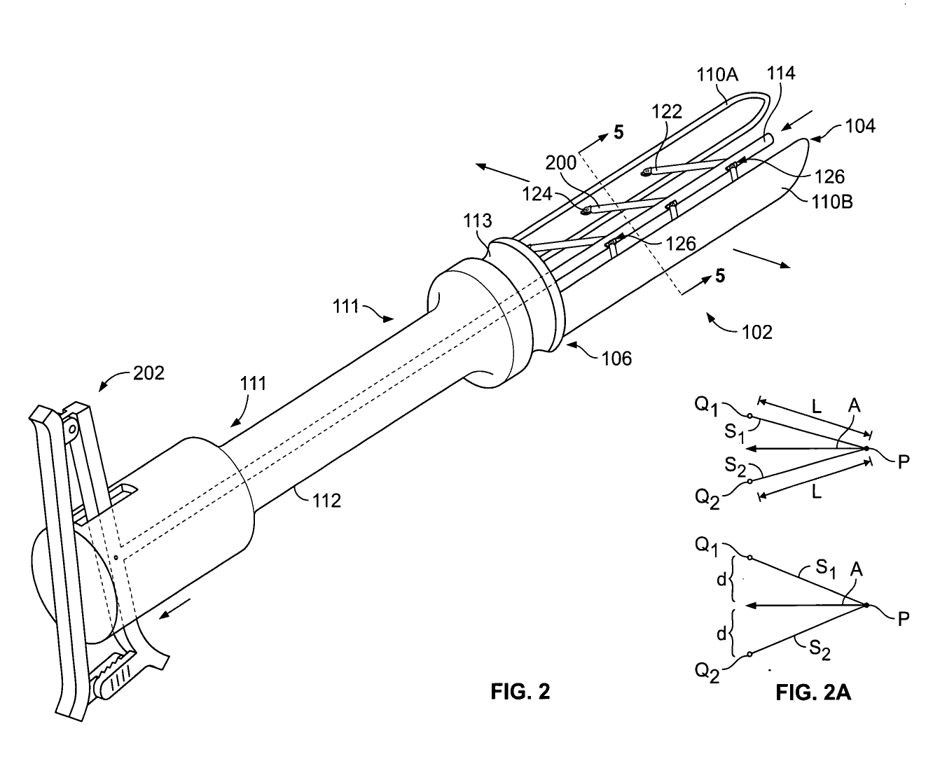

FIG. 2 is a perspective view of Maasal dilator 100 but with the contoured deflection panels 110A and 110B of the malecot 110 radially displaced from an elongated control rod 114 that itself has a central axis coincident with the longitudinal axis 108 of the dilating element 102 and the insertion depth limiter 112. The deflection panels 110A and 110B are radially displaced away from the control rod 114 and the longitudinal axis 108 by the deflection of the several swing arms 122 that extend between the control rod 114.

Each of the swing arms 122 are rigid rods or tubes that have first and second opposing ends 124 and 126 that are each pivotally connected to the deflection panels 110 and control rod 114 respectively. Since the swing arms 122 are rigid, they are virtually incompressible such that a radial force exerted on the end attached to the back side of the deflection panels 110A and 110B will cause the swing arms 122 to rotate about the opposite end, i.e., the end attached to the control rod. Thus, if the angle between the swing arms 122 and the control rod 114 is greater than zero, the application of an axial force on the control rod 114 that causes the control rod 114 to move toward the handle 202 will create an axial force down the length of the swing arms 122 but it will also induce a radial force at the swing arm's point of attachment at the panels 110A and 110B.

The axial force exerted on the swing arms 122 by retraction of the control 114 toward the handle 202 will cause the deflection panels 110A and 110B to butt up against the shoulder 118 of the insertion depth limiter 112. The radial force, however, will urge the panels outwardly, causing them to push against the cervix, in turn causing the cervix to dilate. In one embodiment, three swing arms are used to deflect each panel 110: one arm at each of the distal and proximal ends and one arm between the proximal and distal ends to prevent the panel 110 from bending and to prevent “waisting” of the panels to insure equal dilation along the whole length of the cervix.

Radial displacement of the panels 110 relative to the control rod 114 (as well as the central axis 108) is accomplished through the control rod 114. In the embodiment shown in FIGS. 1, 2 and 3, translational movement of the control rod 114 along the central axis 108 causes the panels 110A and 110B to deflect outwardly.

FIG. 2A shows how the longitudinal movement of the control rod 114 from right to left, or “toward” the proximal end 106 of the panels 110 (as well as toward the control handle 202) will cause the panels 110 to deflect outwardly and radially away from the control rod 114. Thus radial displacement and radial expansion of the malecot 110 can be effectuated and controlled by a linear displacement (or linear movement) of the control rod relative to the insertion depth limiter 112 and the panels 110.

In FIG. 2A the vector A depicts movement of the control rod 114 vis-à-vis the swing arms 122. The two fixed-length line segments S1 and S2 represent two swing arms, each of them having a common end P and opposing ends Q1 and Q2.

As can be seen in FIG. 2A, the radial distance between the ends Q1 and Q2 and the line representing vector A is denoted in FIG. 2A as “d.” The distance “d” is equal to the length of the line segments S1 or S2, which is “L”, multiplied by the sine of the angle θ formed between the line segments and the vector A. The equation is written as:

d=(L)·sin θ (1)

Since the total radial displacement distance between two panels 110A and 110B is equal to 2d, for every angle θ, there will be a corresponding total radial expansion or displacement equal to 2L sin θ, where d=L sin θ.

Those of ordinary skill in the art will recognize that since “d” is equal to L multiplied by sin θ, the lateral translation of the control rod 114 for any value of the panel expansion “d,” relative to a fixed point, such as the insertion depth limiter, is equal to L, multiplied by cosine θ. Therefore, for every radial displacement of 2L sin θ, the corresponding linear displacement of the control rod will be L cos θ. Thus, in an alternate embodiment, control rod 114 is marked with displacement indicator lines 115 as shown in FIGS. 3, 6 and 7, which can be used to directly display radial displacement or opening of the malecot's panels 110A and 110B relative to each other or relative to the control rod 114 central axis. As set forth above, the spacing between the lines 115 on the control rod or other indicator rod will be non-linear, when using a swing-arm transmission described above.

Referring again to FIG. 2A, it can be seen that as the vector A moves to the left in FIG. 2A, the angle θ will increase and “d” will increase. It is also important to note, however, that if the angle θ is zero or less than zero, the axial force exerted on the line segments S1 and S2 by the force represented by vector A will be entirely compressive, i.e., no radial force will be exerted at points Q1 and Q2. If no radial force is exerted at Q1 and Q2, neither of them will move away from the vector A. Thus, it is important that the swing arms 122 form a non-zero angle with the longitudinal axis 108 when the panels 110A and 110B are in their fully-retracted position in order to insure that a radial force will be exerted on the swing arms 122 and hence on the panels 110A and 110B.

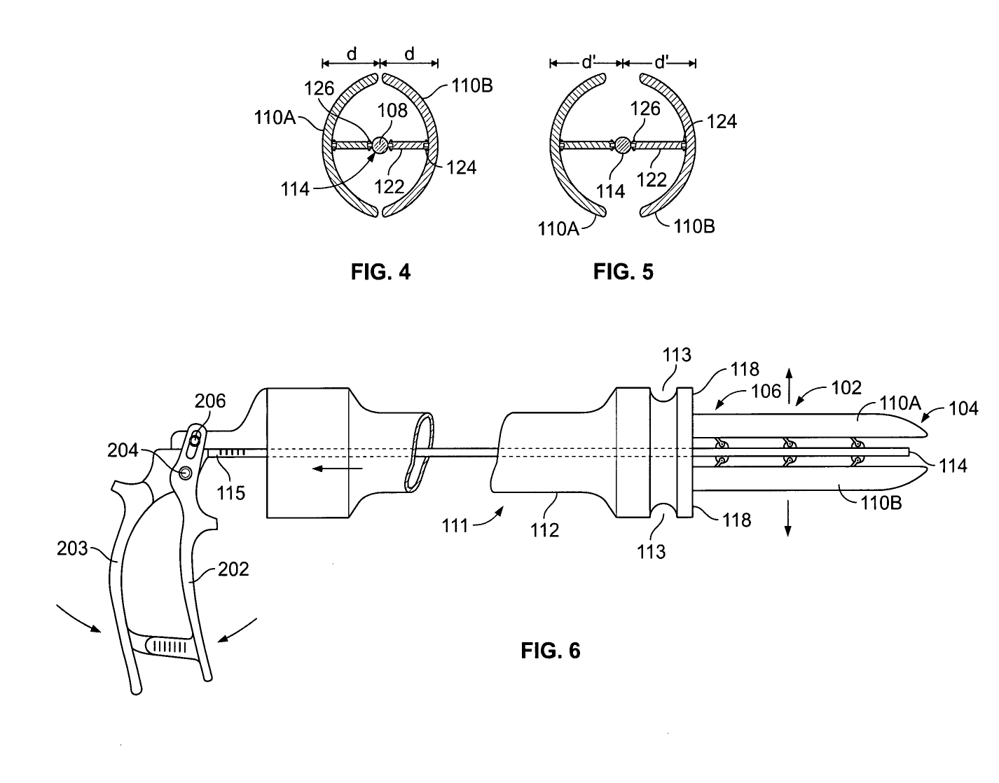

FIG. 4 shows an end view of the dilating element 102, taken along the section lines 4-4 shown in FIG. 1. In FIG. 4, the deflection panels are 110A and 110B fully retracted and the distance “d” is small.

FIG. 5 shows the same end view of the dilating element 102, albeit with the deflection panels radially displaced from the control rod 114. As can be seen in FIG. 4 and FIG. 5, the contoured panels 110A and 110B that form the malecot 110 are radially expandable relative to the longitudinal axis 108 and the control rod 114. Those of ordinary skill in the art will see that FIGS. 1, 2, 2A demonstrate that the radial displacement (2d′) of the panels 110A and 110B, which form the malecot 110 will be a function of the length of the swing arms 122 and the linear movement of the control rod along the longitudinal axis 108.

At least one significant advantage of the Maasal cervical dilator 100 over the prior art is that cervical dilation can be precisely controlled. Another significant advantage is that penetration depth of the malecot 110 is limited to be the length of the two elongated and contoured panels 110A and 110B by the shoulder 118 of the insertion depth limiter 112.

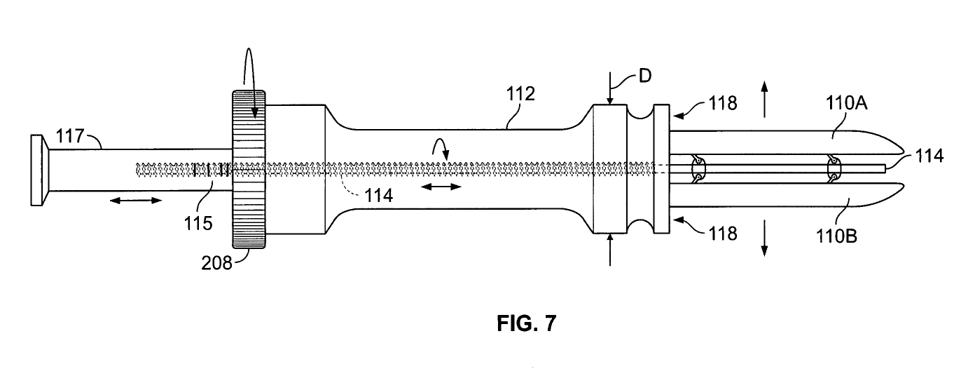

Referring again to FIG. 1 and FIG. 2, the insertion depth limiter 112 is essentially a tube or cylinder having an outside diameter at the end next to the panels 110A and 110B that is large enough so that it cannot be inserted into the cervical canal. At the proximal end of the malecot 110, the insertion depth limiter 112 the shoulder 118 diameter (See the outside diameter “D” in FIG. 7.) is large enough to prevent it from being accidentally inserted into the cervical canal. Since the insertion depth limiter 112 is too large in diameter to enter the cervix or the canal, perforation of the uterus by the malecot 110 is prevented.

FIG. 3 shows a diagrammatic representation of the human cervical canal 101. FIG. 3 also shows the malecot 110 and its contoured panels 110A and 110B inserted into the canal 101. The embodiment of the insertion depth limiter 112 shown in FIG. 3 has an outside diameter that blocks the limiter 112 from entering the canal 101. The distance or space between the outside diameter and the exterior surfaces of the malecot 110 form a shoulder 118. The shoulder 118 abuts the opening of the cervical canal.

In a preferred embodiment the length of the panels 110A and 110B is approximately 5 cm. The outside diameter of the insertion depth limiter 112 is at least 2 cm., in order to prevent the insertion depth limiter 112 from entering the cervix. The outside diameter of the collapsed or closed malecot 110 is preferably less than 4 mm and preferably 3-3.5 mm. At the malecot's 110 maximum displacement, the spacing between the contoured panels 110A and 110 is preferably about 2 cm. Because of anatomical differences and because some cervix' may be partially dilated for a various reasons, alternate embodiments also include malecots having a collapsed or closed outside diameter of 1 cm. or more.

FIG. 6 shows a cross-sectional view of the dilating element 102 and a partial cut away of the insertion depth limiter 112. FIG. 6 also shows the circumferential groove 113 that accepts a binding ring of an elastic cover (not shown) and the narrowed grip region 111. More importantly, however, FIG. 6 shows additional detail of a transmission mechanism 200 that both couples the control rod 114 to the malecot 110 and which controls a linear movement of the control rod 114 so as to cause the malecot 110 to displaced radially.

In FIG. 6, two operator handles 202 and 203 cooperate to effectuate translation of the control rod 114 with respect to the malecot 110, the panels 110A and 110B, and the insertion depth limiter 112. An axis or pivot point 204 allows one of the handles 202 or 203, to pivot or rotate around the axis 204 and displace the control rod 114. Those of ordinary skill in the art will recognize that either one of the handles or both handles can be configured to move the control rod 114 along its axis. When the control rod 114 is moved toward the handles 202 and 203, the panels 110A and 110B will butt up against the shoulder 118 but they will also be urged outwardly and away from the control rod 114.

FIGS. 1, 2 and 6 depict one implementation of a transmission mechanism 200 that couples the control rod to the malecot 110 such that linear movement of the control rod 114 along the longitudinal axis 108 causes the malecot 110 to displaced radially. FIG. 7 shows another embodiment of a transmission mechanism.

In FIG. 7, the transmission mechanism 200 includes a knurled thumb nut 208, the interior of which is threaded to mate with exterior threads on the control rod 114. As the thumb nut 208 rotates clockwise or counterclockwise in the handle of the maasal cervical dilator 100, it causes the control rod 114 (having an exterior thread that mates with and engages threads inside of the thumb nut 208) to translate linearly. In the embodiment shown, the threaded control rod 114 retracts into a handle 117. The handle 117 is provided with a window through which the deflection scale markings 115 can be seen. When the control rod 114 moves along the axis 108 by the rotation of the thumb nut 208, the contoured panels 110A and 110B that comprise of the malecot 110 will displaced radially with respect to the axis 108.

In the embodiment shown in FIG. 7, rotation of the thumb nut 208 will cause radial displacement of the panels 110A and 110B and hence the enlargement i.e. displacement of the malecot 110. The thumb nut 208 can also be attached to the control rod 114 and rotably mounted to the insertion depth limiter 112 such that the control rod 114 rotates with the fixed thumb nut 208. In such an alternate embodiment, the swing arms are pivotally attached to freely-rotating nuts that are threaded onto the control rod (not shown) in order to allow the control rod to rotate within the nuts to which the swing arms are attached. In such an embodiment, rotation of the thumb nut 208 will cause the swing arms to translate along the axis 108, however, the control rod 114 will not.

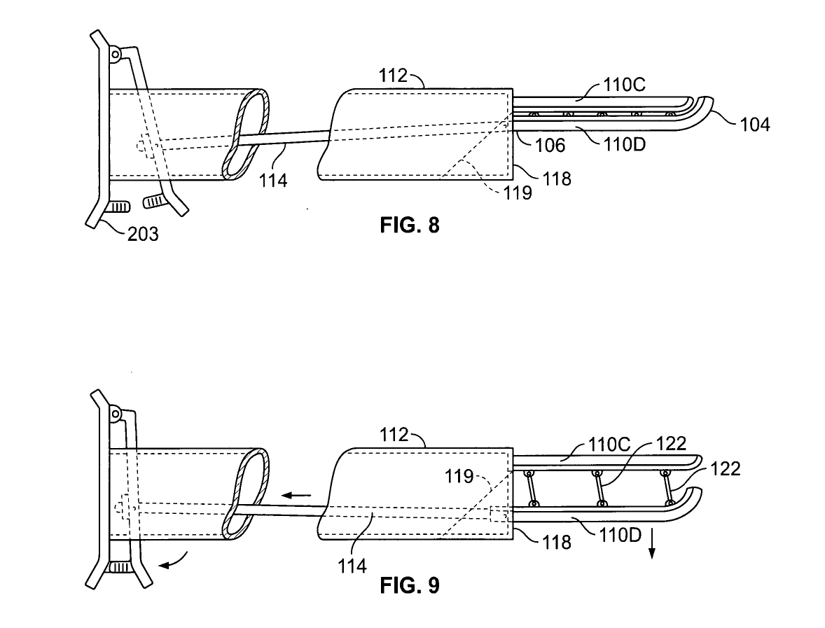

FIGS. 8 and 9 depict another embodiment of a transmission mechanism 200 that couples the control rod 114 to the malecot 1110 such that linear movement of the control rod 114 will cause the malecot 110 contoured panels to displaced radially. In these figures, one panel 110C is fixed to the insertion depth limiter 112 but the other panel 110D is free to move radially and laterally.

As the control rod 114 is pulled to the left, i.e., toward the handle 203, the proximal end 106 of the moveable panel 110D slides outwardly as it slides “up” or away from the fixed panel 110C, on a ramped slot 119 formed into the shoulder 118 of the insertion depth limiter 112. As the proximal end 106 slides “up” the ramped slot 119 under the force exerted by the control rod 114, the panel 110D also slides away from the distal end toward the handle 203.

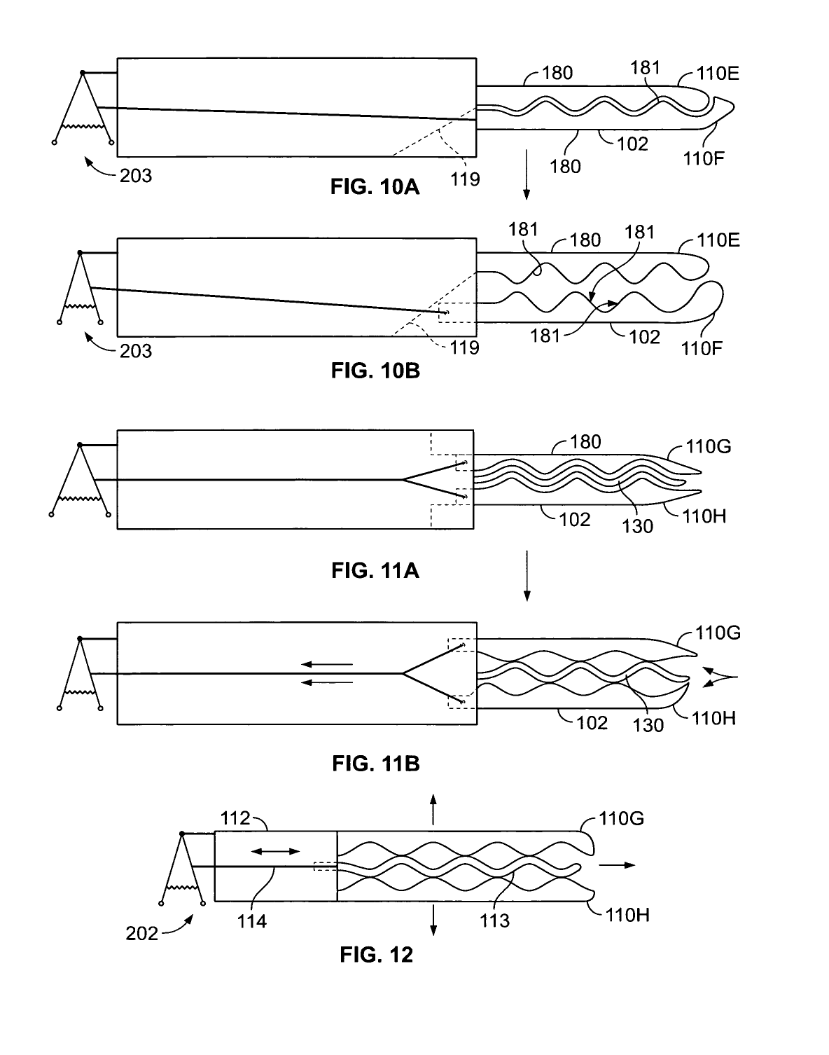

FIG. 10A and FIG. 10B show yet another embodiment of a maasal cervical dilator. Panels 110E and 110F have a smooth side 180 facing outwardly or away from the control rod 114 and a wavy, sinusoidal or boustrophedonic side 181 that faces inwardly and toward the control rod 114. In the collapsed or compressed state shown in FIG. 10A, the boustrophedonic sides 181 nest together. The panel 110E is fixed to the shoulder of the insertion depth limiter 112; the other panel 110F is slidably coupled to an inclined ramp 119 cut into the shoulder 118. Translational movement of the opposite panel 110F toward the handle 203 will cause the second panel 110F to ride “up” the slot ramp 119 but also radially away from the first panel 110E as shown in FIG. 110B. Thus, by moving one panel 110F through the lateral translation of the control rod 114, the distance between the smooth sides 180 can be controllably increased.

FIG. 11A and FIG. 11B show a variation of the embodiment shown in FIGS. 10A and 10B. In FIGS. 11A and 11B, the panels 110G and 110H have a smooth side 180 facing outwardly as in FIGS. 10A and 10B. They also both have a wavy, sinusoidal or boustrophedonic side 181 that faces inwardly and toward the control rod 114, as in FIGS. 10A and 10B. In FIGS. 11A and 11B, however, both of the panels 110G and 110H are translated relative to a fixed central deflection panel 113, both sides of which are boustrophedonic such that the panels 110G, 110H and the deflection panel 113 can all be nested together in a collapsed state shown in FIG. 11A. In FIG. 11B, however, both of the panels 110G and 110H are pulled into slots formed in the shoulder 118. As the panels 110G and 110H move relative to the central deflection panel 130, both the panels are urged away from the panel 130 by the crests of the boustrophedonic surfaces.

In yet another embodiment and as shown in FIG. 12, the panels in FIGS. 11A and 11B are held in place and the central deflection panel 113 is translated laterally relative to the panels 110G and 110H to cause the panels to separate.

Those of ordinary skill in the art will appreciate that the various structures shown in the figures are controllable dilation devices that perform at least the function of controllably dilating a cervix, and importantly, while the device is inside the cervix. Two contoured panels make up the malecot in the embodiments shown in the figures, however, alternate and equivalent embodiments include three, four or more such panels, provided that additional panels are appropriately driven by the control rod. In the embodiments, shown, lateral or rotational movement of a control rod causes the radial deflection of at least one panel, relative to the control rod and/or a second panel, which can also be fixed or movable.

The structure shown in the figures and identified by reference numeral 112 is an insertion depth limiting device that performs a function of limiting the insertion of any kind of malecot into a cervical canal, whether the malecot is expandable or not. By limiting insertion depth, trauma to the uterus can be avoided.

In each of the foregoing embodiments, it is important to note that the distal end 104 of the dilating element 102 is rounded or blunt, to facilitate insertion of the dilating element into the cervical canal. Blunting or rounding the end of the distal end 104 of the dilating element 102 will also reduce the likelihood of cervical canal injury as the dilating element 102 is inserted into the canal.

In yet another embodiment, the malecot panels 110 and perhaps part of the insertion depth limiter 112 can be wrapped in an elastic prophylactic cover, similar to a condom in order to further minimize trauma. Such a cover can also facilitate removal of the device from the cervix. The groove 113 at the proximal end of the insertion depth limiter 112 can be gripped by an elastic band formed into an elastic prophylactic cover placed over the maasal cervical dilation device but which is not shown for clarity.

From the foregoing, those of ordinary skill in the art will appreciate that Maasal cervical dilator 100 provides a closely controlled dilation mechanism by which a physician or other caregiver can more carefully and less painfully effectuate cervical dilation without having to use multiple different devices, which the prior art requires. While the device shown above is straight, alternate embodiments of the invention include dilators having malecot panels 110A and 110B that are curved or articulated to accommodate anatomical differences. In addition, the insertion depth limiter 112 can also be curved or articulated, itself or relative to the malecot panels to accommodate anatomical differences.

Those of ordinary skill in the art will also appreciate that the maasal dilator 100 described above and shown in the figures can be assembled from injection molded plastic pieces, the manufacturing cost of which is quite low. By assembling such a device it is feasible to construct a single-use dilator, the sterility of which could be more reliably assured. An alternate embodiment of the cervix dilator 100 described above could also be assembled from stainless steel or other durable materials that could be re-used.

The foregoing description and the illustrations in the various figures are all examples of preferred embodiments. The true scope of the invention described herein is set forth in the appurtenant claims.

What is claimed is:

1. A maasal cervical dilator comprising:
a dilating element for insertion into a cervical canal, said dilating element having a distal end and a proximal end separated from each other along a longitudinal axis of the dilating element, the dilating element further comprising an expandable malecot; and
an insertion depth limiter, sized, shaped and arranged to limit the depth by which the malecot can be inserted into the cervical canal.

2. The maasal cervical dilator of claim 1, wherein said malecot is comprised of at least first and second elongated and contoured panels.

3. The maasal cervical dilator of claim 2, further comprising: an elongated central control rod having an axis substantially coincident with the longitudinal axis, the control rod being movable relative to the dilating element such that movement of the control rod causes the at least first and second panels to move away from the control in a direction that is radial to the control rod.

4. The maasal cervical dilator of claim 2, wherein the control rod is configured such that linear movement of the control rod along the longitudinal axis causes the at least first and second panels to separate from each other.

5. The maasal cervical dilator of claim 2, wherein rotational movement of the control rod about the longitudinal axis causes the at least first and second panels to separate from each other.

6. The maasal cervical dilator of claim 1, wherein the malecot is comprised of at least one panel, coupled to the control rod by at least one transmission mechanism that causes the radial separation of the at least one panel from the control rod by movement of the control rod.

7. The maasal cervical dilator of claim 1, wherein the malecot is comprised of at least first and second panels, coupled to the control rod by at least one transmission mechanism that causes the at least first and second panels to radially separate from the control rod by movement of the control rod.

8. The maasal cervical dilator of claim 2 wherein said transmission mechanism transfers a longitudinal motion, rotational motion, or a combination thereof to said panels.

9. The maasal cervical dilator of claim 1, wherein the insertion depth limiter is comprised of a shoulder extending away from the longitudinal axis radially and substantially orthogonally, by a distance sufficient to prevent the proximal end of the dilation element from entering the cervical canal.

10. The maasal cervical dilator of claim 1, wherein the insertion depth limiter is comprised of an annular shoulder of a cylinder, substantially centered about the longitudinal axis and extending substantially orthogonally from the axis, the annular shoulder having an outside diameter greater than a dilated diameter of the cervix.

11. The maasal cervical dilator of claim 1, wherein the insertion depth limiter is further comprised of a circumferential groove.

12. The maasal cervical dilator of claim 1, wherein the first distance is substantially equal to the cervix canal's length.

13. The maasal cervical dilator of claim 4, wherein said transmission mechanism comprises at least one of:
a threaded rod and nut;

14. The maasal cervical dilator of claim 4 wherein said transmission mechanism converts and transfers a rotational motion of said dial to said dilating element.

15. The maasal cervical dilator of claim 1, further comprising a scale that indicates the separation of the panels.

16. The maasal cervical dilator of claim 3, wherein said control rod is provided with a scale that indicates the separation of the panels.

17. The maasal cervical dilator of claim 1, wherein said malecot and said insertion depth limiter are articulated relative to each other.

18. The maasal cervical dilator of claim 1, wherein at least one of said malecot and said insertion depth limiter are curved.

19. The maasal cervical dilator of claim 2, wherein said control rod is flexible.

20. The maasal cervical dilator of claim 1, wherein at least one of the malecot and the insertion depth limiter is plastic.

21. The maasal cervical dilator of claim 1, wherein at least one of the malecot and the insertion depth limiter is stainless steel.

22. The maasal cervical dilator of claim 1, further comprising a protective cover over the panels.

23. A cervical dilator comprising:
an expandable dilating element for insertion into the cervical canal of the of the uterus, said expandable dilating element having at least first and second panels, each of which has a distal end for insertion into the cervical canal and further having a proximal end, the distal and proximal ends being separated from each other along a longitudinal axis of the dilating element, the panels comprising a limited-deflection, radially-expandable malecot; and
an insertion depth limiter located along the longitudinal axis and spaced apart from the distal end by a first distance, the insertion depth limiter limiting insertion of the dilating element into the cervical canal to substantially the first distance.

24. The cervical dilator of claim 23, further comprising: an elongated central control rod having an axis coincident with the longitudinal axis, the control rod being movable in order to cause the at least first and second panels to expand outward from the longitudinal axis.

25. The cervical dilator of claim 24 further including a transmission mechanism that couples the control rod to the panels such that linear movement of the control rod along the longitudinal axis causes the at least first and second panels to displace radially.

26. The cervical dilator of claim 24, further including a transmission mechanism that couples the control rod to the malecot such that rotational movement of the control rod about the longitudinal axis causes the at least first and second panels to displace radially.

27. The cervical dilator of claim 23 wherein the insertion depth limiter is comprised of a shoulder extending away from the longitudinal axis radially and substantially orthogonally, the shoulder having a diameter sufficient to prevent the proximal end of the dilation element from entering the cervical canal.

28. The cervical dilator of claim 23, wherein the insertion depth limiter is comprised of a shoulder that extends substantially orthogonally from the axis, the shoulder having an outside diameter greater than a dilated diameter of the cervix.

29. The cervical dilator of claim 26, wherein said transmission mechanism comprises at least one of:
a threaded rod and nut;

30. The cervical dilator of claim 26 wherein said transmission mechanism converts and transfers a rotational motion of said dial to said dilating element.

31. The cervical dilator of claim 26, further comprising a scale indicating the distance by which the at least first and second panels are separated.

32. A cervical dilator comprising:
an expandable dilator means for controllably dilating a cervix and
an insertion depth limiter means for limiting the insertion of the expandable dilator means.

33. The cervical dilator of claim 32 further comprising a transmission means for causing the expansion of the expandable dilator means by a lateral translation relative to the insertion depth limiter.

34. The cervical dilator of claim 32 further comprising a transmission means for causing the radial enlargement of the expandable dilator means by a lateral translation of a control rod extending through the expandable dilation means.

35. A cervical dilator comprising:
a first panel having a first smooth side and a first wavy side; and
a second panel having a second smooth side facing away from the first smooth side and further having a second wavy side that faces the first wavy side;
wherein, lateral translation of the first contoured panel relative to the second contoured panel causes the distance between the first and second smooth sides to increase.

36. The cervical dilator of claim 35, further comprising an insertion depth limiter, which limits the depth to which the first and second panels can be inserted into a cervical canal.

37. The cervical dilator of claim 35 wherein the insertion depth limiter includes an inclined ramp on which at least one of the first and second panels slides.

38. A cervical dilator comprising:
a first panel having a first smooth side and a first wavy side; and
a second panel having a second smooth side facing away from the first smooth side and further having a second wavy side that faces the first wavy side;
a wavy deflection panel, between the first panel and second panel, the lateral translation of which, relative to at least one of the first and second panels causes the distance between the first and second panels to increase.

39. The cervical dilator of claim 38, further comprising an insertion depth limiter, which limits the depth to which the first and second panels can be inserted into a cervical canal.

40. The cervix dilator of claim 39, wherein the insertion depth limiter is comprised of an annular shoulder substantially centered about the longitudinal axis and extending substantially orthogonally from the axis, the annular shoulder having an outside diameter greater than a dilated diameter of the cervix.

41. The dilation system of claim 38 further comprising a transmission mechanism that converts and transfers at least one of: a lateral translation and a rotational motion, into a radial deflection of at least one of the first and second panels.

42. The cervical dilator of claim 38 wherein the insertion depth limiter includes an inclined ramp on which at least one of the first and second panels slides.
